(19) United States
(12) Patent Application Publication
(10) Pub. No.: US 2009/0227923 A1
(43) Pub. Date: Sep. 10, 2009

(54) COOLING SYSTEM FOR ORTHOPEDIC CAST

(76) Inventors: Christopher A. Markus, Westfield, IN (US); Jeanne S. Martin, Noblesville, IN (US)

Correspondence Address: ICE MILLER LLP, ONE AMERICAN SQUARE, SUITE 3100, INDIANAPOLIS, IN, 46282-0200, US

(21) Appl. No.: 12/044,630

(22) Filed: Mar. 7, 2008

(51) Int. Cl. A61F 5/00; A61F 7/00

(52) U.S. Cl. 602/2; 602/14; 607/114; 607/96

(57) ABSTRACT

A therapy system, device and method comprising a wrap adapted to be secured to the body surface of a user. The wrap further comprises at least one wire and a plug that is capable of removably coupling with an external cooling device. The external cooling device is coupled with the wrap by a cord and the plug to deliver a cooling effect to the body surface, thereby removing heat therefrom. When the therapy is complete, the wrap may be easily removed from the body surface through cutting or similar means, and is entirely disposable.

### FIELD

The field generally relates to orthopedics and a system and method for controlling the temperature of a body surface contained within an orthopedic device.

### BACKGROUND

The treatment of broken bones or trauma often involves the use of a wrap or cast whereby the site of injury is stabilized and/or supported for healing purposes. Conventionally, such devices are comprised of orthopedic bandages or other flexible materials used in conjunction with a settable composition. The bandages function as a buffering fabric and comprise cotton or other flexible material. The settable composition is typically a curable liquid resin that is capable of hardening upon activation by a solvent, heat or irradiation (e.g., Plaster of Paris or fiberglass). When applied to an injured appendage, the flexible materials are first wrapped around the injured area to form a flexible sleeve. Thereafter, the settable composition is applied to the outer portion of the flexible sleeve and allowed to harden, thereby immobilizing the injured limb contained therein.

While the hardened cast provides sufficient rigidity to support the limb, conventional casts are often uncomfortable to wear because they retain heat and cause discomfort such as itching. Further, the trauma itself often manifests itself in the form of swelling which results from the accumulation of bodily fluids underlying the skin adjacent to the site of the trauma. When the patient is wearing a hardened cast to support the area, such swelling not only results in patient discomfort, but also inhibits recovery, as the cast results in the increased application of pressure against the tissue and surrounding nerve and organ structures. Conventional hardened casts do not facilitate the reduction of swelling as such casts are not meant to be easily removed and do not expand to alleviate the increased pressure caused by such swelling. In an attempt to remedy the discomforts provided by casts of the prior art, it is known to use porous or woven base materials for the flexible sleeve. This, however, has not proven ideal because the interstices of the woven material are blocked by the curable composition when it is applied to the exterior of the flexible sleeve.

Alternatively, other types of conventional casts attempt to actively cool the body part within the cast. Several mechanisms have been developed to achieve this goal, including the application of cold compresses such as ice packs for prescribed periods of time to the site of trauma. This method has met with only limited success as the cold compresses are oftentimes initially too cold for the patient to comfortably endure and, as a result, the patient is unable to tolerate the cooling effects of the compresses for the prescribed period of time. Furthermore, because the compress removes heat from the body, the temperature of the compress itself progressively increases, thereby diminishing its temperature reducing affects. Such methods also require that the cast be removed for a period of time while the cold compress is in place, thereby leaving the site of trauma unsupported and vulnerable when the compress is in use.

It is further known to incorporate tubes into the interior of a cast such that cold fluid can be pumped therethrough to cool the limb. This method has also not proven ideal for several reasons. Primarily, the tubes must be of a sufficient diameter to allow fluid to flow therethrough, which is uncomfortable to the patient due to the resultant pressure on the injured limb within the cast. In addition, such systems require the patient to be connected to a fluid reservoir, and such systems have a tendency to leak; two undesirable characteristics given that exposure to moisture can reduce the integrity and strength of the cast. Such cast systems are also rather costly to manufacture and are not easily removed once the treatment is completed. Accordingly, it is desirable to produce a cast and therapy system that is capable of reducing the temperature of the encased body part in order to reduce swelling and to provide a more comfortable experience to the patient.

### SUMMARY

In one embodiment, a wrap comprises a first inner layer, at least one wire and an outer layer. The first inner layer is formed from a flexible material and is configured to conform to the body surface of the user. At least one wire is thereafter applied to the body surface on top of the first inner layer. Once the first inner layer and the at least one wire are applied, the outer layer is applied, thereby surrounding the first inner layer and the at least one wire. In one embodiment, the outer layer comprises a hardening substance such that the outer layer provides some rigidity and protection to the layers and the body part encased therein. The wrap further comprises a plug coupled to the outer layer of the wrap. The plug is in electrical communication with the at least one wire encased within the outer layer.

In one embodiment of a therapy system, the therapy system includes the above-described wrap and an external cooling device. The external cooling device is capable of generating a cooling effect that can be transferred to the interior of the wrap. An electrical cord is coupled with the external cooling device and may be used to transmit the cooling effect to the wrap. When the plug is mounted on the outer layer of the wrap, the electrical cord of the external cooling device can be removably coupled therewith and used to transmit the cooling effect to the interior of the wrap. In this manner, the cooling effect generated by the external cooling device is transferred to the encased body party through the at least one wire of the wrap.

In an alternative embodiment, the external cooling device comprises a Peltier thermoelectric device having a plurality of conductors and a heat sink. Further, an external power supply is coupled with the external cooling device and provides sufficient power to run the external cooling device and generate the cooling effect. The external cooling device and the power supply can be combined into a single electronics package that is portable and separate from the wrap.

The therapy system and wrap described herein may be used to treat an injured body part of a patient. In one embodiment, a therapy system is provided comprising a wrap having a first inner layer, at least one wire and an outer layer. The therapy system also comprises an external cooling device having at least one Peltier thermoelectric device, a power supply, and an electrical cord. The power supply can either be housed within the same electronics package as the Peltier thermoelectric devices, or it can be separate, such as a wall outlet or battery source. Once the wrap is secured to the patient's injured body part as previously described above, the wrap is removably coupled with the external cooling device by way of the electrical cord. The external cooling device generates a cooling effect, which is transferred through the electrical cord and the at least one wire to the body part encased within the wrap. Because the Peltier thermoelectric devices are housed within an external cooling device, the resultant heat generated through the operation of the Peltier thermoelectric device is concentrated in the heat sink and is dissipated away from the patient.

### BRIEF DESCRIPTION OF THE DRAWINGS

FIG. 1 shows schematic view of one embodiment of a wrap applied to a target body surface.

FIG. 2A shows a cross-sectional view of the wrap of FIG. 1 taken along line A-A of FIG. 1.

FIG. 2B shows a cross-sectional view of the wrap of FIG. 1 taken along line B-B of FIG. 1.

FIG. 3 shows a cross-sectional view of another embodiment of a wrap applied to a target body surface.

FIG. 4 shows a schematic view of one embodiment of a therapy system comprising the wrap of FIG. 1.

FIG. 5 shows a schematic view of another embodiment of a therapy system comprising the wrap of FIG. 1.

### DETAILED DESCRIPTION

Reference will now be made to the embodiments illustrated in the drawings and specific language will be used to describe the same. It will nevertheless be understood that no limitation of scope is intended by the description of these embodiments.

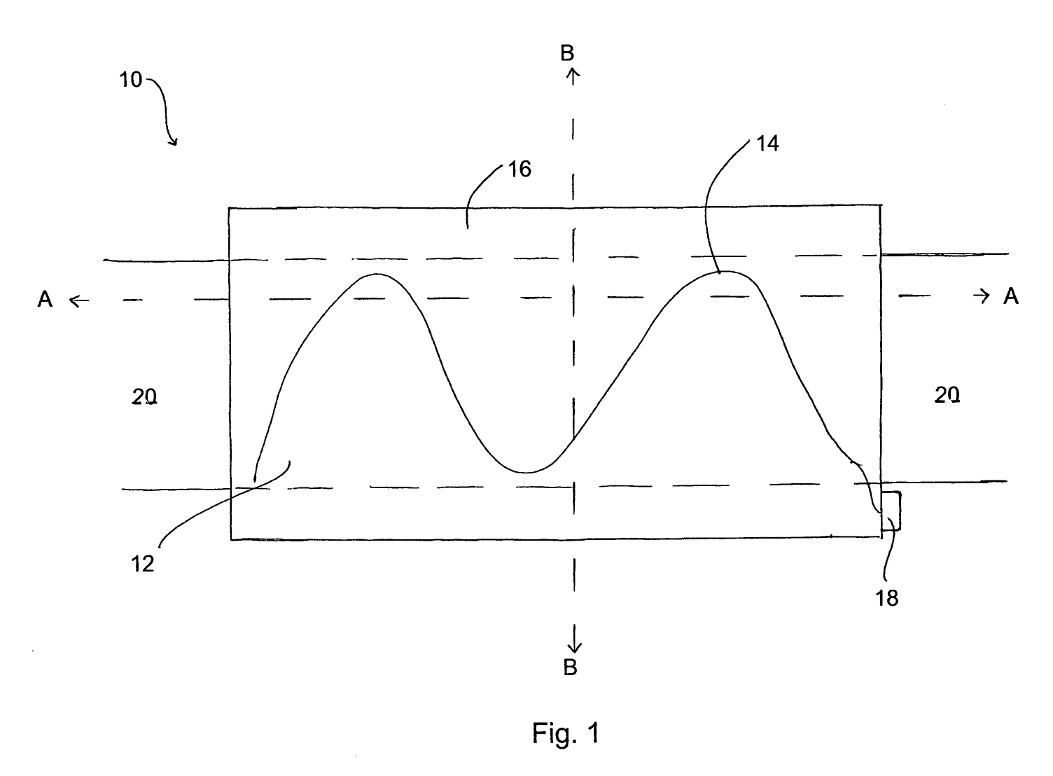

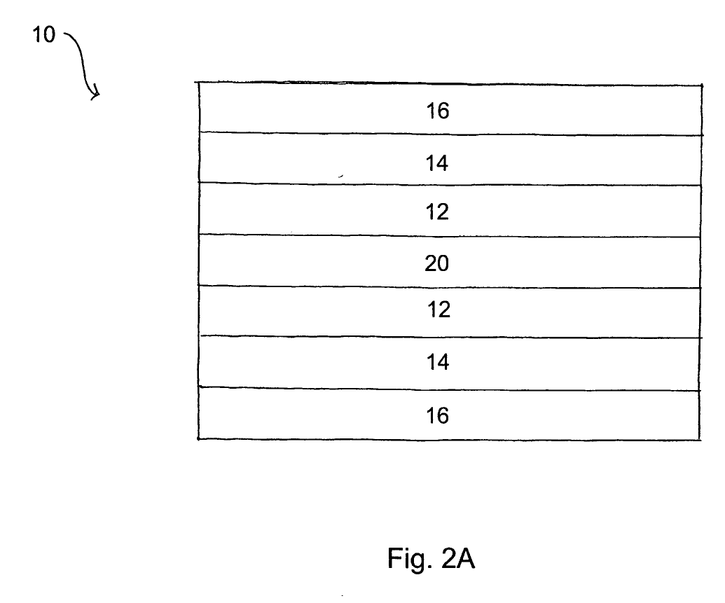

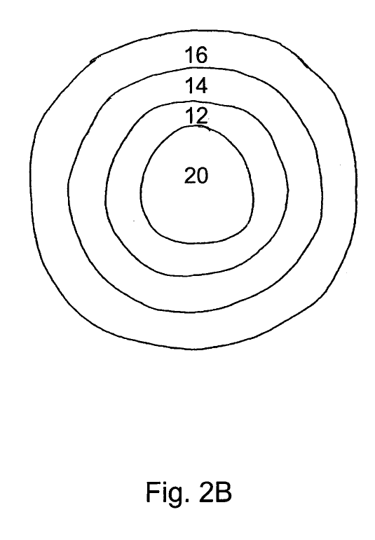

FIGS. 1, 2A, and 2B show one embodiment of a wrap 10 applied to a target body surface 20. Specifically, FIG. 1 shows a side view of the wrap 10. FIG. 2A shows a cross-sectional view of the wrap 10 of FIG. 1 taken along the line A-A of FIG. 1. FIG. 2B shows a cross-sectional view of the wrap 10 of FIG. 1 taken along the line B-B of FIG. 1. In this embodiment, the wrap 10 comprises an orthopedic cast that is capable of cooling the body part to which it is attached. However, it will be recognized that the wrap 10 may comprise any item that may be worn or carried on a user's body, including bandages, harnesses, dressings, splints, or any other type of dressing that may be applied to an area of trauma. The wrap 10 is adapted to be secured to a target body surface 20, which may include, for example and without limitation, any part of a body such as a wrist, neck, arm, knee, or leg. The wrap 10 may further be formed in various shapes to fit the contours of the target body surface 20. For example, wrist and knee wraps comprise rectangular shaped strips that can encircle the appropriate body part, whereas a shoulder wrap includes a sleeve area for mounting over the patient's shoulder and down the patient's arm. Further, more than one wrap 10 may be applied to an individual at a time and therefore may be used to treat multiple injury sites.

In the embodiment shown in FIGS. 1, 2A, and 2B, the wrap 10 has a first inner layer 12, at least one wire 14, an outer layer 16, and a plug 18. The first inner layer 12 comprises a flexible material capable of conforming to the target body surface 20 to which the wrap 10 is attached. For example, the first inner layer 12 may comprise a thermally conductive material such as conductive mesh, a ventilated cushion material, a cotton material, a synthetic material, a buffering fabric, or any material that is capable of being shaped to contact and envelope the target body surface 20. When the first inner layer 12 is wrapped around the injured area, it forms a flexible sleeve that is in direct contact with the target body surface 20. In one embodiment, the first inner layer 12 comprises strips of material that are wrapped and fitted around the target body surface 20. In an alternative embodiment, the first inner layer 12 is preformed into a flexible sleeve, such that the first inner layer 12 immediately fits snuggly and comfortably around the target body surface 20, thereby reducing the amount of time required for application.

The outer layer 16 of the wrap 10 encases the first inner layer 12, generally does not directly contact the underlying target body surface 20 and, as is known in the art, may comprise a settable composition. For example, the outer layer 16 may be initially applied to the inner layer 12 in a series of wet, flexible strips that harden upon drying to form an orthopedic cast. Alternatively, the outer layer 16 may be pre-formed to correspond to the shape of the inner layer 12 and/or the target body surface 20 such that when the outer layer 16 is secured to the inner layer 12 it immediately provides rigidity to the wrap 10.

The outer layer 16 may comprise Plaster of Paris, fiberglass, plastic, neoprene or any other material that is capable of providing some degree of rigidity to the wrap 10. It will be appreciated that the degree of rigidity present in the outer layer 16 may vary depending on the preferred treatment for a particular ailment. In one embodiment, the outer layer 16 functions to substantially immobilize the target body surface 20 and provide protection and support thereto. In an alternative embodiment, the outer layer 16 exhibits minimal rigidity and is relatively flexible and soft to provide functional stabilization and variable support to the target body surface 20. By altering the rigidity of the outer layer 16, the wrap 10 can be used to effectively treat broken bones, bone fractures or soft tissue injuries.

The outer layer 16 of the wrap 10 further comprises a plug 18 coupled therewith. In the embodiment shown in FIG. 1, the plug 18 is positioned adjacent to the target body surface in a convenient manner; however, it will be appreciated that the plug 18 may be positioned on any portion of the outer layer 16. The plug 18 has a first end and a second end and can comprise any type of electrical connector or conductive device that is capable of receiving electrical current from a conductor. The first end of the plug 18 comprises a mating surface 30. The mating surface 30 may be a female socket or male plug configured to respectively receive or be received by a conductive device. The second end of the plug is embedded within the outer layer 16 and is configured to couple with the at least one wire 14 such that the plug 18 and the at least one wire 14 are in electrical communication.

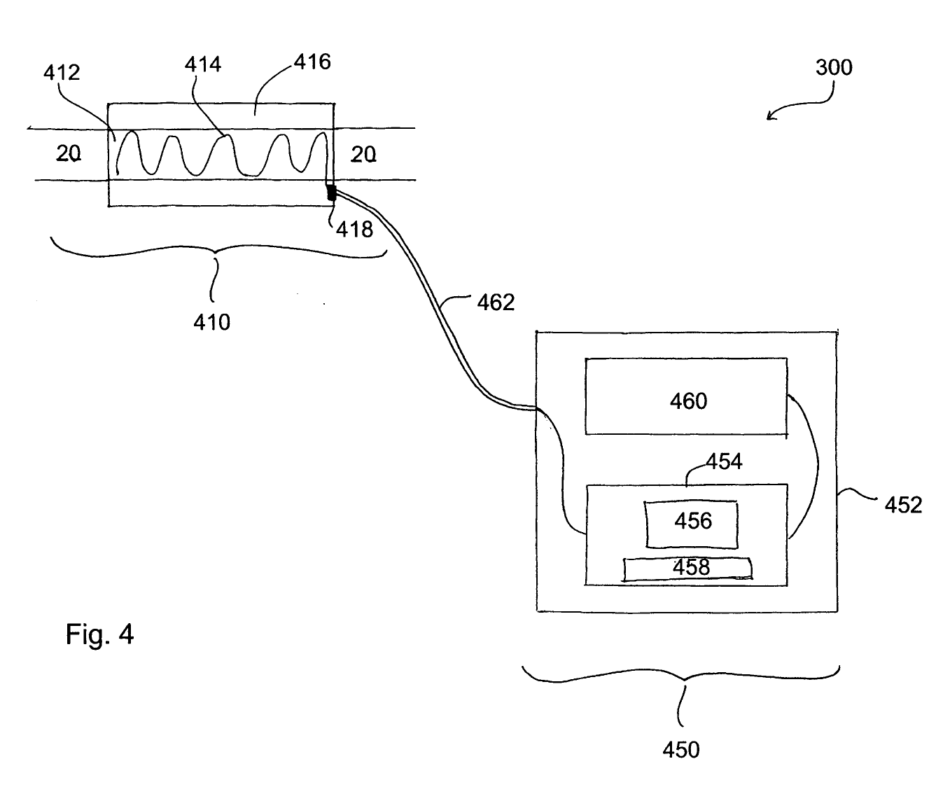

In the embodiment shown in FIG. 1, the first end of the plug 18 protrudes from the surface of the outer layer 16 of the wrap 10, and the second end of the plug 18 is positioned slightly within the outer layer 16. In this manner, the mating surface 30 of the first end of the plug 18 is positioned to receive a conductive device. Alternatively, the body of the plug 18 may be encompassed within the outer layer 16 such that only a minimal portion of the first end of the plug 18 protrudes from the outer layer 16. Still further, the body of the plug 18 may be wholly encompassed within the outer layer 16 such that the mating surface 30 of the first end of the plug 18 is flush with the exterior of the outer layer 16 (see FIG. 4).

The arrangement of the wrap 10 further comprises the at least one wire 14 that is capable of transmitting a cooling effect to the target body surface 20. The at least one wire 14 is positioned between the first inner layer 12 and the outer layer 16 of the wrap 10 and comprises relatively thin, flexible, conductive members. The at least one wire 14 can be configured in any manner so long as the various parts and/or pieces of the wire 14 are in electrical communication with the plug 18. For example, the conductive members of the at least one wire 14 may be independent elements that stem from a common trunk, or the conductive members may be configured into one continuous strand. Further, the at least one wire 14 may be arranged in a specific pattern and embedded into a mesh. It will be appreciated that the most effective configuration of the at least one wire 14 will depend on the nature of the injury to be treated and the location of the target body surface 20.

Referring back to FIGS. 1, 2A, and 2B, the at least one wire 14 extends substantially along the length of the first inner layer 12 so as to be positioned in close proximity with the target body surface 20. In the embodiment shown in FIG. 2A, the at least one wire 14 is coupled with the first inner layer 12 in such a manner that the at least one wire 14 does not directly contact the target body surface 20. In an alternative embodiment, the at least one wire 14 is secured to the first inner layer 12 in a suitable manner, such as by way of electrodeposition. In yet another embodiment, the at least one wire 14 is embedded within the first inner layer 12, thereby minimizing the thickness of the wrap 10.

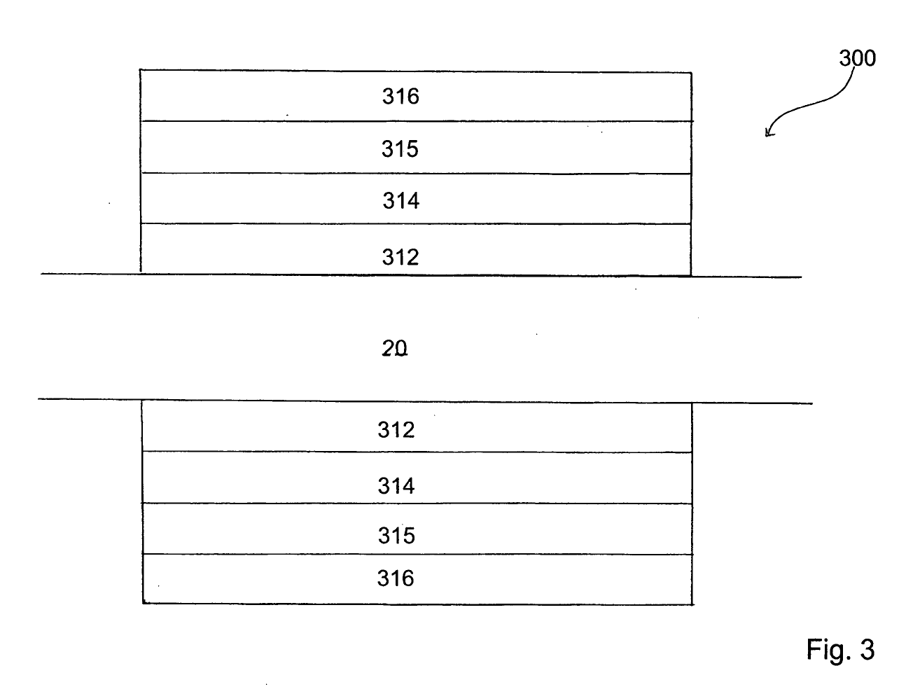

FIG. 3 shows an alternative embodiment of the wrap 10 of FIGS. 1, 2A, and 2B. The wrap 300 in FIG. 3 comprises a first inner layer 312, at least one wire 314, a second inner layer 315, and an outer layer 316. It will be recognized that the only difference between the wrap 300 and the wrap 10 is the addition of a second inner layer 315. Depending on the particular trauma that is being treated by the wrap 300, the second inner layer 315 may comprise any type and thickness of layer(s) deemed necessary, provided the outer layer 316 may be applied thereon. In this manner, a user can control the thickness, density and rigidity of the wrap 300. Because the second inner layer 315 is not positioned in between the at least one wire 314 and the target body surface 20, the second inner layer 315 does not substantially effect the transmission of cooling effect from the at least one wire 314 to the target body surface 20.

Now referring to FIG. 4, a schematic view of one embodiment of a cast cooling system 400 is shown. The cast cooling system 400 comprises wrap 410, which is identical to the wrap 10 of FIGS. 1, 2A, and 2B. Similar to wrap 10, wrap 410 comprises a first inner layer 412, at least one wire 414, and outer layer 416, and a plug 418. The cast cooling system 400 further comprises an external cooling unit 450. As shown in FIG. 4, the external cooling unit 450 comprises a cooling device 454 and a power supply 460, both housed within a housing 452. The housing 452 provides protection to the components housed therein and may be any housing unit known in the art. In one embodiment, the housing 452 is stationary and configured to rest in one location. In an alternative embodiment, the housing 452 is mobile, such that the housing may be easily maneuvered by the user. For example, the housing 452 may have wheels attached thereto or comprise any other mechanism capable of facilitating the mobility of the housing 452. Accordingly, because the power supply 460 is contained within the housing 452, the external cooling unit 450 may be portable and transferred by the user at their convenience.

As previously described, the housing 452 contains the cooling device 454. The cooling device 454 may comprise any cooling device known in the art that is capable of producing a cooling effect transmittable through a conductive material. In the embodiment shown in FIG. 4, the cooling device 454 comprises a Peltier thermoelectric device having at least two conductors 456 and a heat sink 458.

Generally, the Peltier thermoelectric device is a solid-state, active heat pump that transfers heat from one side of the device to the other against a temperature gradient (from cold to hot). The conductors 456 of the Peltier thermoelectric device are comprised of dissimilar materials connected to each other at least two junctions. Due to these junctions and the use of dissimilar materials, when the Peltier thermoelectric device is connected to a DC voltage source, the current drives a transfer of heat from one junction to the other. In this manner, the Peltier thermoelectric device causes one side to cool, while the other side heats up. Accordingly, the Peltier thermoelectric device achieves heat transfer through the consumption of electrical energy.

The cooling device 454 obtains the requisite electrical energy from the power supply 460. In the embodiment shown in FIG. 4, the power supply 460 is contained within the housing 452 and is electronically coupled the cooling device 454. The power supply 460 may be any power supply that is capable of producing a sufficient amount of energy to support the functionality of the cooling device 454. In the embodiment shown in FIG. 4, the power supply 460 is of the type that generates DC voltage.

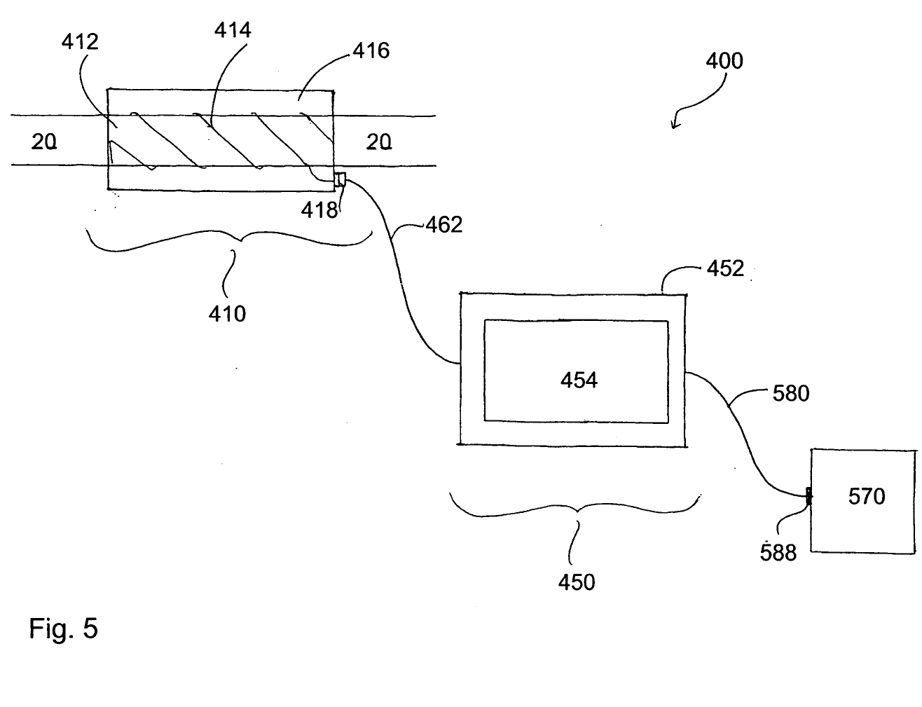

In an alternative embodiment, the power supply may be independent of the housing 452 and the external cooling device 450. Now referring to FIG. 5, wherein identical numerals indicate identical parts, a schematic view of an alternative embodiment of the therapy system 400 is shown. In this embodiment, a power supply 570 is positioned outside of the housing 452 and the housing 542 only contains the cooling device 454 and any other electrical components necessary to generate and transmit a cooling effect. Further, the housing 452 comprises a power cord 580 extending therefrom. The end of the power cord 580 that is not attached to the housing 452 comprises a detachable coupling 588 configured to mate with the external power supply 570. The external power supply 570 may be any power supply known in the art, such as a wall outlet, generator, or battery source. In one embodiment, the detachable coupling 588 is configured to couple with a wall outlet such that the cooling device 554 is portable and convenient to use. Further, it will be recognized that any combination of power supplies may be used in connection with the therapy system 400. For example, a power supply 460 may be housed within the housing 452 and an external power supply 570 may be removably coupled with the housing 452 by power cord 580.

Now referring back to FIG. 4, the external cooling unit 450 further comprises an electrical cord 462 that extends from the housing 452 and is in electrical communication with both the cooling device 454 and the power supply 460. The electrical cord 462 comprises any cord that is capable of transmitting electrical current and has a first end and a second end. The first end of the electrical cord 462 is coupled with the housing 452, thereby forming an electrical connection with the cooling device 454 contained therein. The second end of the electrical cord 462 comprises a detachable coupling that is configured to removably mate with the mating surface 430 of the plug 418. When the detachable coupling of the electrical cord is mated with the mating surface 430 of the plug 418, the electrical cord 462 provides an electrical connection between the external cooling unit 450 and the wrap 410. In this manner, the at least one wire 414 is in electrical communication with the cooling device 454 and the power supply 460 of the external cooling unit 450.

The wrap 410 is applied by securing the various layers thereof around the target body surface 20. It will be recognized that the layers of the wrap 410 may be applied to the target body surface 20 in any order so long as the outer layer 416 comprises the outermost component. For example, in one embodiment, the first inner layer 412 is applied first so that the first inner layer 412 is in direct contact with the target body surface 20. Thereafter the at least one wire 414 is applied over the first inner layer 412. In an alternative embodiment, the at least one wire 414 is embedded within the first inner layer 412 and the first inner layer 412 and the at least one wire 414 are applied in tandem.

After the first inner layer 412 and the at least one wire 414 are applied, the plug 418 is positioned in such a manner that the plug 418 can be easily coupled with the outer layer 416 of the wrap 10. Further, the mating surface 430 of the plug 418 is positioned to be exposed to the exterior of the wrap 410. Thereafter, any number of filler layers may be wrapped around the first layer 412 and the at least one wire 414 (e.g., second inner layer(s) 315). Such filler layers may comprise cushioning for the patient's comfort, or functional layers for compression to increase the support provided to trauma site.

Once all of the interior layers have been wrapped around the target body surface 20, the outer layer 416 is applied. The exact method used to apply the outer layer 416 will depend upon what type of material selected to form the outer layer 416. For example, when the outer layer 416 comprises Plaster of Paris, a bandage impregnated with plaster is moistened and wrapped around the interior layers of the wrap that have been previously applied to the target body surface 20. Thereafter, the bandage is allowed to set and harden, thereby forming a hard, protective coating. In an alternative embodiment, the outer layer 416 comprises a neoprene bandage, which is fitted over the previously applied interior layers. Thereafter, straps are employed to secure the neoprene-based outer layer 416 in place. Irrespective of the type of material selected for the outer layer 416, the plug 418 is embedded within the outer layer 416 such that the mating surface 430 is positioned to receive the detachable coupling of the electrical cord 462. The exact placement of the plug 418 in the outer layer 416 is dependent upon the particular requirements of the injury at issue.

In operation, the cast cooling system 400 can be used to treat an area of trauma by supporting the injured area and concurrently reducing the temperature thereof to facilitate the reduction of swelling, pain or discomfort in the area. Further, because the external cooling unit 450 is removably coupled with the wrap 410, the cooling therapy can be applied in a convenient, non-intrusive manner. For example, when the patient is experiencing pain or discomfort to the area of trauma, he or she can simply plug the external cooling unit 450 into the wrap 410 to initiate the cooling therapy. When the therapy session is complete, the patient simply unplugs the wrap 410 from the external cooling unit 450 and returns to his or her normal activities, unhindered by any extraneous equipment.

The cooling effect is generated by the external cooling unit 450 and transferred to the target body surface 20 by the at least one wire 414. As previously indicated, when the cooling device 454 operates, the current flowing through it has two effects: cooling and heating. Depending in the direction of the current flowing through the conductors 456, the cooling effect and the heating effect can be transmitted in different directions. Accordingly, current is applied to the cooling device 454 in such a manner so that the cooling effect is transmitted through the electrical cord 462 to the at least one wire 414, and the heating effect is channeled to the heat sink 458. In this manner, the at least one wire 414 within the wrap 414 cools the target body surface 20 without removing the wrap 410.

As noted above, the unwanted heating effect generated by the cooling device 454 is accumulated in the heat sink 458 and allowed to dissipate into the surrounding atmosphere. The heat sink 458 is located within the housing 452 and, as such, the heat is dispelled away from the user. Accordingly, the heat released from the heat sink 458 is not proximate to the user and therefore does not interfere with the cooling therapy. While the methods described herein are illustrated using a Peltier device as the cooling device 454, it will be recognized that any cooling device may be used, provided the cooling device is capable of transferring the cooling effect to the at least one wire 414 within the wrap 410 through the electrical cord 462.

When the treatment to the user's body part is complete and/or it is necessary to remove the wrap 410 from the target body surface 20, the wrap 410 may be removed using methods known in the art. For example, a cast saw may be used to simply cut the wrap 410 from the user's body part. The various layers of the wrap 410, including the at least one wire 414, are easily cut through and removed. Thereafter, the entire wrap 410 may be discarded and replaced if necessary. It will be recognized that the application and removal of the wrap 410 can be completed by efficient processes that are known in the art.

The embodiments described herein are only offered by way of non-limiting examples, as other versions are possible. It is anticipated that a variety of other modifications and obvious changes will be apparent to those having ordinary skill in the art and such modifications and changes are intended to be encompassed within this description and the following and any later added claims.

1. A therapy system comprising:
a wrap adapted to be secured around a body surface, the wrap comprising:
a first inner layer comprising a flexible material configured to conform to the body surface,
at least one wire positioned adjacent to the first inner layer and configured to conform to the body surface, and
an outer layer surrounding the first inner layer and the at least one wire; and
an external cooling device removably coupled with the at least one wire of the wrap, wherein the external cooling device is capable generating a cooling effect that may be transferred through the at least one wire.

2. The therapy system of claim 1, wherein the outer layer of the wrap comprises a rigid material adapted to immobilize a body part.

3. The therapy system of claim 2, wherein the wrap comprises an orthopedic cast.

4. The therapy system of claim 1, wherein the first inner layer further comprises a sleeve.

5. The therapy system of claim 1, wherein the at least one wire is disposed on a mesh material positioned between the first inner layer and the outer layer.

6. The therapy system of claim 1, wherein the at least one wire is embedded within the first inner layer.

7. The therapy system of claim 5, further comprising a second inner layer disposed between the outer layer and the at least one wire, and configured to conform to the body surface.

8. The therapy system of claim 1, wherein the at least one wire is disposed in a serpentine pattern.

9. The therapy system of claim 1, wherein the external cooling device comprises a first cord having a mating surface, the mating surface of the first cord capable of removably coupling with the at least one wire such that, when coupled, the first cord and the at least one wire are in electrical communication.

10. The therapy system of claim 9, further comprising a plug coupled with the outer layer and comprising a mating surface, the plug being in electrical communication with the at least one wire and the mating surface of the plug capable of removably coupling with the mating surface of the first cord.

11. The therapy system of claim 10, wherein at least the mating surface of the plug protrudes from the outer layer.

12. The therapy system of claim 1, wherein the external cooling device comprises at least one Peltier thermoelectric device having a plurality of conductors and a heat sink.

13. The therapy system of claim 1, wherein the external cooling device comprises an internal power supply in electrical communication with the Peltier thermoelectric device.

14. The therapy system of claim 1, wherein the external cooling device further comprises a second cord comprising a mating surface, the mating surface of the second cord configured to removably couple with an external power supply.

15. The therapy system of claim 1, wherein the wrap may be easily removed from the body surface.

16. The therapy system of claim 15, wherein the wrap is cuttable.

17. The therapy system of claim 16, wherein the at least one wire is cuttable.

18. The therapy system of claim 15, wherein the wrap is disposable.

19. A therapy system comprising:
a wrap adapted to be secured around a body surface, the wrap comprising:
a first inner layer comprising a flexible material configured to conform to the body surface,
at least one wire embedded within the first inner layer,
an outer layer surrounding the first inner layer and the at least one wire, and
a plug coupled with the outer layer and comprising a mating surface, the plug being in electrical communication with the at least one wire,
an external cooling device removably coupled with the at least one wire of the wrap, the external cooling device comprising:
at least one Peltier thermoelectric device having a plurality of conductors and a heat sink,
at least one power supply in electrical communication with the at least one Peltier thermoelectric device, and
a cord having a mating surface, the mating surface of the cord capable of removably coupling with the mating surface of the plug such that, when coupled, the cord and the at least one wire are in electrical communication,
wherein the external cooling device is capable of generating a cooling effect, which may be transferred to a body surface through the at least one wire.

20. A method of treating an injured body part of a patient, the steps comprising:
providing a system comprising:
a wrap adapted to be secured around a body surface, the wrap comprising:
a first inner layer configured to conform to the body surface,
at least one wire coupled with the first inner layer,
an outer layer surrounding the first inner layer and the at least one wire, and
a plug coupled with the outer layer and comprising a mating surface, the plug being in electrical communication with the at least one wire; and
an external cooling device removably coupled with the at least one wire of the wrap and comprising:
at least one Peltier thermoelectric device having a plurality of conductors and a heat sink,
at least one power supply in electrical communication with the at least one Peltier thermoelectric device, and
a cord having a mating surface, the mating surface of the cord capable of removably coupling with the mating surface of the plug such that, when coupled, the cord and the at least one wire are in electrical communication;
applying the wrap around a body part;
removably coupling the mating surface of the cord with the mating surface with the plug; and
using the external cooling device to generate the cooling effect proximate to the body part within the wrap.

21. A wrap comprising:
a first inner layer configured to conform to an external body surface;
at least one wire embedded within the first inner layer and removably coupled with an external cooling device; and
an outer layer surrounding the first inner layer and the at least one wire;
wherein the external cooling device comprising a heat sink, the external cooling device capable of producing a cooling effect that may be transmitted through the at least one wire to the external body surface.
